(19) United States
(12) Patent Application Publication
(10) Pub. No.: US 2006/0282134 A1
(43) Pub. Date: Dec. 14, 2006

(54) Photo-thermal therapeutic device

(76) Inventors: Ronald S. Shapiro, Toledo, OH (US); Richard C. Dunlap, Holland, OH (US); David B. Sutton, Monroe, MI (US)

Correspondence Address: EMCH, SCHAFFER, SCHAUB & PORCELLO CO, P O BOX 916, ONE SEAGATE SUITE 1980, TOLEDO, OH, 43697, US

(21) Appl. No.: 11/437,070

(22) Filed: May 19, 2006

Related U.S. Application Data
(60) Provisional application No. 60/689,185, filed on Jun. 10, 2005.

(51) Int. Cl. A61N 5/06; A61F 7/00; A61F 7/12

(52) U.S. Cl. 607/88; 607/96

(57) ABSTRACT

A photo-thermal therapeutic device having an outer housing that can be used to treat patients. A plurality of light emitting diodes and resistors are resiliently mounted in the housing. The resilient mounting of the diodes and resistors allow the device to conform and provide effective treatment of almost any surface of the body of a user.

### This application claims the benefit of U.S. provisional patent application Ser. No. 60/689,185 filed Jun. 10, 2005.

### BACKGROUND OF THE INVENTION

The present invention relates to photo-thermal therapeutic devices and more specifically to such a device which incorporates a plurality of light emitting diodes (LEDs) protruding from a housing. The diode resiliently mounted in the housing to accommodate treatment of almost any surface of the body for an exceedingly wide range of the population.

The use of light and heat to provide musculoskeletal pain relief, promote cosmetic rejuvenation, promote accelerated healing of open and closed wounds as well as numerous other benefits has long been known. However, devices for use in such treatments have in general been designed for specific applications or been relatively cumbersome to use.

### SUMMARY OF THE INVENTION

The present invention, however, provides a uniquely designed device for use in photo-thermal treatments which is easy to use while also being capable of adapting to most body contours for a wide range of individuals while still providing effective photo-thermal treatment. The housing includes a plurality of LEDs arranged in spaced relationship along a resilient surface. Additionally, the LEDs are preferably designed for variable sequential firing so as to resist body adaptation to the treatment which adaptation may significantly and progressively reduce the effectiveness of the treatment over time.

The photo-thermal device utilizes energy from both infrared non-coherent light and resistive and latent diode heat to produce its dramatic effects by accelerating the naturally occurring reparative mechanisms of the organism. The physio-chemical effects of heat (thermal energy) have been recognized in the health care field. When the specialized photo-thermal energy is delivered to the cell there is interaction between the light and the cells. This is referred to as “photomodulation”, and there is evidence that photomodulation accelerates wound healing.

Exposure to the photo-thermal energy produced by the instant invention results in acceleration of collagen synthesis and deposition. Procollagen production is increased resulting in enhanced collagen synthesis through selective action on collagen gene expression at the transcriptional level. This results in a more rapid increase in wound tensile strength, thereby decreasing the likelihood of wound dehiscence. Application of the photo-thermal device stimulates macrophages (a type of white blood cell) to release factors that stimulate fibroblast replication and proliferation (e.g. monokines). Cellular effects that occur include mitochondrial hyperplasia, the appearance of cytoplasmic microfilament bundles, and the deposition of an abundant fibrillar matrix in pericellular regions. Treatment with the device also accelerates the formation of a functional scar. Energy delivered by the photo-thermal device is absorbed at the mitochondrial level and is available for photochemical reactions, which leads to an increase in oxidative enzyme activities. Photon absorption by cytochromes, which are present in large numbers in mitochondrial crests, results in an enhancement of protein synthesis producing increased substance for wound repair.

Health Care Professionals have observed that treatment with the invention accelerates epitheliazation across open wounds. This speeds the healing of the wound, and also reduces the risk of secondary infection, which would delay healing time and increase morbidity. The use of the invention on a patient increases local nitric oxide (NO) concentration, which stimulates anabolic responses. The presence of nitric oxide in the wound promotes a cytotoxic effect in the defense against viruses, bacteria, fungi, and parasites. Keeping a wound free of infection is an important factor in minimizing delayed wound healing. The photo-thermal device facilitates reduction of wound edema and this allows earlier and more complete approximation of the epithelial edges of the wound which further enhances the rate of wound healing. All the above natural healing mechanisms including the chemical, cellular, and subcellular processes are accelerated through biomodulation when exposed to photo-thermal energy.

Application of the photo-thermal device to the surface of painful soft tissue areas and open and closed wounds results in an increase in seratonin production and other neurotransmitter substances. Seratonin is a chemical precursor to endorphins, enkephalins, and dinorphins and subsequently increased levels of these naturally occurring “morphine like” products circulate in the body resulting in a reduction in pain by binding to opiate receptors. Use of the device of the present invention diminishes localized edema (swelling) in the affected area; a less swollen and less tense area becomes less painful.

Application of the device on a patient, relaxes painful reflex muscle spasm by restoring normal muscle tissue properties on a cellular level through adenosine triphosphate (ATP) formation and enzyme activityⁱ Relaxation of spastic muscles relieves the painful stimulation and irritation of A and C nerve fibers. Treatment with this device decreases sensory nerve conduction velocity, and raises the perception threshold of sensory nerves which promotes pain reduction. Application of the device with a patient can variably raise skin temperature, which produces local comfort, vasodilatation, increased blood flow. These factors contribute to relaxation of muscle spasm, pain relief, and accelerated healing.

Additional advantages and features of the present invention will become apparent from the subsequent description and the appended claims taken in conjunction with the accompanying drawings.

### BRIEF DESCRIPTION OF THE DRAWINGS

FIG. 1 is a cross sectional view of the photo-thermal device of the present invention.

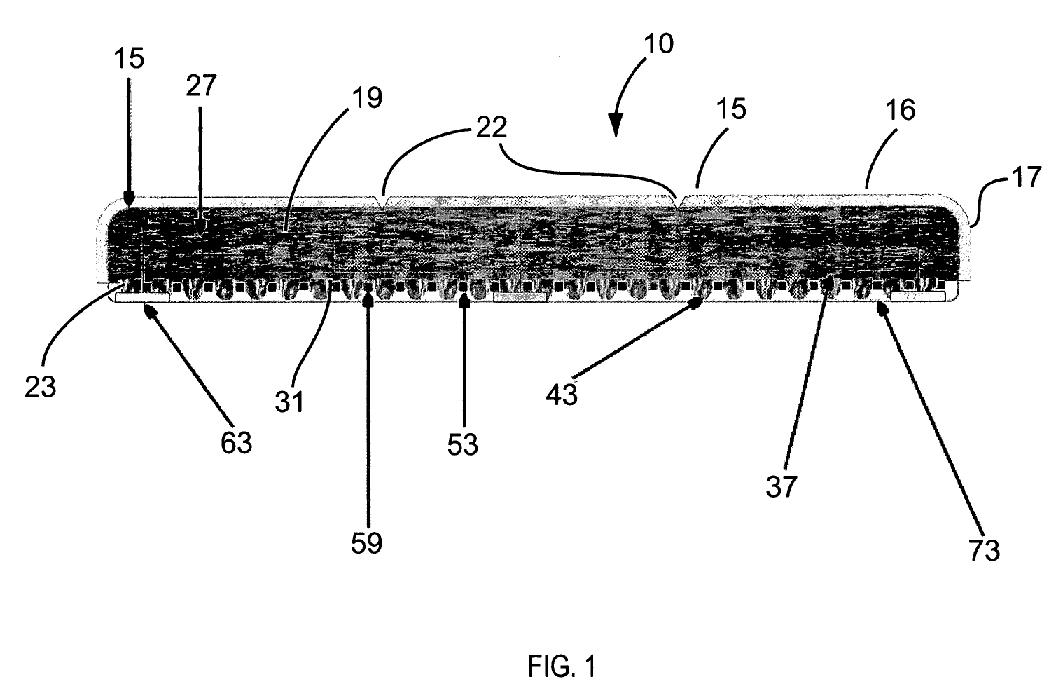

FIG. 2 is a plan view of the photo-thermal device.

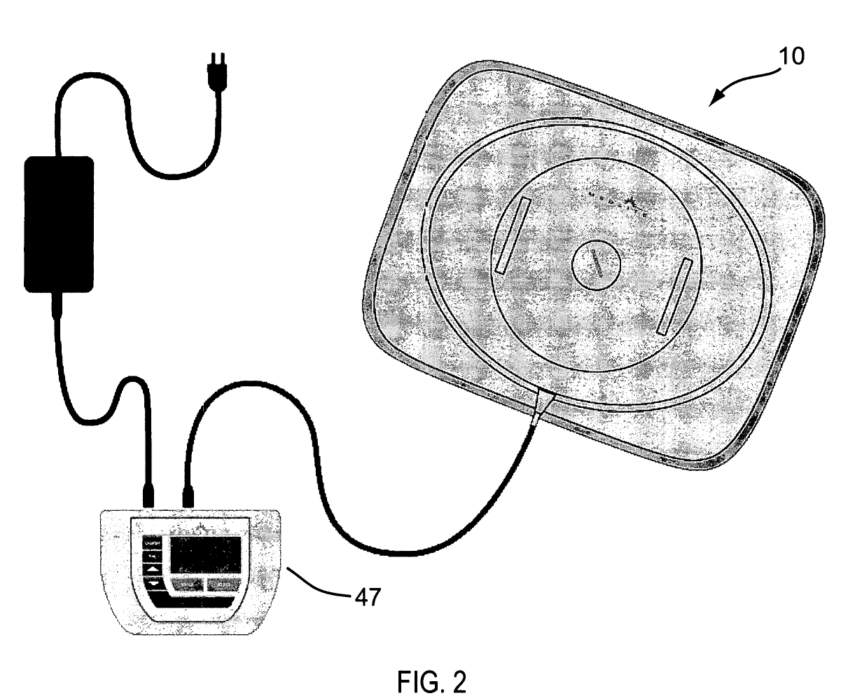

FIG. 3 is a perspective view of the back of the photo-thermal device.

FIG. 4 is a perspective view of the back of the device of FIG. 3 with the securing mechanism removed.

FIG. 5 is a top view of the securing device show in FIG. 3.

FIG. 6 is a bottom view of the securing device shown in FIG. 5.

### DETAILED DESCRIPTION OF THE PREFERRED EMBODIMENT(S)

The invention is directed to a photo-thermal treatment device 10 as shown in the attached drawings. Photo-thermal treatment devices have been previously developed by the Assignee of this patent application. The teachings of U.S. Pat. No. 6,187,029 owned by the Assignee of this application are hereby specifically incorporated by reference as part of this patent application. The device is intended for noninvasive neurovascular stimulation in a user or patient. The photo-thermal device has an outer housing 15 having a back wall 16 and a sidewall 17 that defines a cavity 19. An opening 23 is positioned on the side of the outer housing 15 opposite the back wall 16 and the opening is in communication with the cavity 19. The outer housing 15 is designed to be positioned adjacent to the surface that is to receive light and heat stimulation from the photo-thermal device 10. In most applications the device is used with humans and it has been found that an outer housing having a width from about 5 to about 30 inches and a height from about 1 to about 12 inches will be satisfactory to accommodate most users for the photo-thermal device. The cavity 19 has a depth from about 0.5 to about 5 inches and the opening 23 essentially extends between the sidewall 17 of the outer housing 15. However, it should be understood that the opening 23 can be smaller then the cavity 19 formed in the outer housing 15 if desired. If the outer housing is designed to be particularly large, at least one hinge or fold joint 22 can be provided in the cover to facilitate the conforming or contouring of the photo-thermal components positioned in the cover to accommodate different areas of the body. The hinge or joint 22 allows the device 10 to be contoured or shaped and brought into proximity with the body during use.

A resilient material 27 is positioned in the cavity 19 of the outer housing 15. The resilient material 27 is secured by suitable securment means to the back wall 16 and sidewall 17 of the outer housing 15. In practice it has been found that an adhesive type of material can be positioned on the back wall 16 and sidewall 17 that form the cavity 19 to secure the resilient material 27 in the outer housing 15. The resilient material has a surface 31 that is adjacent the opening 23 formed by the outer housing 15. The resilient material 27 is configured to essentially fill the cavity 19. In practice, it has been found desirable to use an easily deformable resilient material. It has also been found that foam materials work particularly well as the resilient material 27 and that foams selected from the group of rubber, vinyl or urethane are particularly well suited for the resilient material 27. In addition, a self-skinning molded foam with selectively cored out areas to enhance the flexibility of the foam in desired locations works particularly well in the photo-thermal device. The resilient material 27 is designed so that it can be displace in both a horizontal and vertical direction. Since the photo-thermal device may not always be used in a manner where horizontal and vertical directions are clearly defined, it should be understood that the resilient material can be displaced in a direction parallel to the back wall 16 and also in a direction that is parallel to the sidewall 17. In fact, the resilient material can be displaced in almost any direction. The resiliency of the material can also vary across the layer of the resilient material to achieve the desired resiliency in specific areas of the material.

Positioned on the surface 31 of the resilient material 27 that is adjacent the opening 23 is a flexible circuit 37. The flexible circuit 37 is secured to the resilient material 27 through the use of an adhesive or other suitable securment means. An example of a suitable flexible circuit 37 is manufactured by Century Circuits. The flexible circuit 37 is connected to a source of electrical energy in a manner that is well known in the art. The flexible circuit 37 is designed so that various electrical components can be connected to the flexible circuit where by electrical energy can be supplied to the various components in a manner to activate and control the electrical components. The flexible circuit 37 is positioned on the resilient material 27 in an orientation that is substantially parallel to the back wall 16 of the outer housing 15. The flexible circuit 37 is positioned to substantially fill the opening 23 defined by the outer housing 15. The flexible circuit is designed so that it can flex in at least two directions and can be displaced in a direction that is parallel to the back wall 16 and in a direction that is parallel to the sidewall 17. In fact, the flexible circuit 37 can simultaneously be displaced or contoured in multiple planes or directions to accommodate the surface area that is being treated. In this manner the flexible circuit 37 is capable of being displaced in essentially the same manner that the resilient material 27 can be displaced and the flexible circuit 37 is capable of conforming to the shape undertaken by the flexible material 27 when the flexible material is displaced. An example of a flexible circuit that is suitable for use with the photo-thermal device of the present invention is manufactured by the Century Circuits Company.

A plurality of light emitting diodes (LED) 43 are operatively connected to the flexible circuit 37 so that the flexible circuit can provide electrical energy to energize the light emitting diodes. An example of a diode that can be used with this device is a SMT diode manufactured by Lumex. The light emitting diodes 43 are positioned on the surface of the flexible circuit 37 that is opposite to the resilient material 27 so that the light emitting diodes 43 extends from the opening 23 defined by the outer housing 15. The light emitting diodes 43 are designed to deliver photo energy and heat to treat a variety of conditions. As the light emitting diodes 43 are mounted on the flexible circuit 37 the light emitting diodes 43 can be displaced along with the flexible circuit 37 and the resilient material 27 to conform to various curvatures and surface contours that are present on the surface that is receiving the light and heat stimulation from the photo-thermal device 10. The light emitting diodes 43 can be positioned on the flexible circuit 37 in a wide variety of patterns or configurations to deliver the desired intensity of photo energy to the user of the photo-thermal device 10.

A controller 47 is operatively connected to flexible circuit 37 for controlling the supply of electrical energy to a variety of electrical components and in particular the light emitting diodes 43. The controller 47 is designed to provide a wide range of operational characteristics for the light emitting diodes 43 including allowing the light emitting diodes to be sequenced in the activated mode to satisfy various operating parameters that will enhance the delivery of photo and thermal energy to the user of the photo-thermal device 10. The controller 47 can control the light emitting diodes 43 so that the light emitting diodes are activated in various geometrical patterns. The controller 47 can also be utilized to establish the rate that each light emitting diode 43 or a cluster of light emitting diodes is electrically activated and deactivated. The pattern and rate at which the light emitting diodes 43 are activated and then deactivated can be controlled by the diode controller 47 to produce the desired treatment results for the user of the photo-thermal device 10. In particular, the controller can provide a variable refresh rate for the photo-thermal device which cycles how often the pattern of the treatment modalities is repeated. An example of a controller 47 that is suitable for use with the flexible circuit 37 is produced by the Pinnacle Technologies Company.

The photo-thermal device 10 has a plurality of resistors 53 operatively connected to the flexible circuit 37 in positioned adjacent the light emitting diodes 43. The Vishay/Dale Company manufactures resistors that can be satisfactorily used in the photo thermal device. The resistors 53 are disposed on the flexible circuit to extend from the opening 23 in the same manner as the light emitting diodes 43. Accordingly, the resistors 53 will come into contact with surface of the user that is to be treated with photo and thermal energy from photo-thermal device 10. The resistors 53 are mounted on the flexible circuit 37 in a manner that the resistors can be displaced in the same manner as the light emitting diodes 43 to accommodate the shape of the surface that is receiving light and heat stimulation from the photo-thermal device 10. The resistors 53 have a heat generating resistive coil that can be activated by the flexible circuit 37 to transmit heat from the resistors 53 to the surface that is being treated. The resistors 53 generate heat energy that can be utilized to augment the thermal energy produced by the light emitting diodes 43. The flexible circuit 37 is configured so that the resistors 53 and the light emitting diodes 43 can be energized at the same time or energized separately. The controller 47 can also be utilized to control the pattern and rate at which the resistors are energized and deenergized by the flexible circuit 37. The pattern and rate for the resistors 53 can established to be essentially the same as the pattern and rate for the light emitting diodes 43 or the controller 47 can be utilized to operate the resistors 53 independently of the pattern and rate of the light emitting diodes 43.

A plurality of thermistors 59 are operatively connected to the flexible circuit 37 and are positioned to be adjacent the light emitting diodes 43 and resistors 53 that extend from the opening 23 defined by the cavity 19. The Texas Instrument Company manufactures thermistors that can be used with this device. The thermistors can be displaced in the same manner as previously described for the light emitting diodes 43 and the resistors 53 to accommodate the contours and shape of the surface that is receiving light and heat stimulation from the photo-thermal device 10. The thermistors 59 are designed to measure the temperature on the surface that is being treated by the photo-thermal device 10. The thermistors 59 are positioned on the flexible circuit 37 in a manner that allows the thermistors to effectively monitor the temperature on the entire surface that is being treated by the photo-thermal device 10. The temperature of the device can be measured segmentally or averaged for the entire surface that is being treated. The flexible circuit 37 contains a feedback loop that reacts to the temperature readings and adjusts the energy supplied to the diodes 43 and resistors 53 to maintain the desired temperature on the surface being treated. The desired temperature for the surface that is being treated can be set with the controller 47 and the thermistor 59 will measure the actual temperature of the surface and supply this information to the controller 47. The actual temperature can be compared to the desired temperature and the controller 47 can adjust the energy supplied to the resistors 53 to maintain the temperature on the surface that is being treated in the desired range. The thermal feedback provided by the thermistors 59 allows the heat energy provided by the surface that is being treated to be maintained at an effective and safe level.

A plurality of electrodes 63 are operatively connected to the flexible circuit 37 and extend from the opening 23 in the same manner as the light emitting diodes 43 and resistors 53 previously described. Electrodes made by The Vishay/Dale Company can be used with the photo-thermal device. The electrodes 63 can be displaced in the same manner as previously described for the light emitting diodes 43 to accommodate the shape of the surface that is receiving light and heat stimulation. The electrodes 63 are utilized to detect the electrical currents that are generated in an active muscle that is receiving treatment from the photo-thermal device 10. The electrical currents that are detected by the electrodes 63 are sent to the controller 47 where an electromyographic instrument can be utilized to evaluate the electrical currents to determine the degree of muscle tension, contraction and relaxation in the muscles that are receiving treatment from the photo-thermal device 10. A graphic display of the degree of muscle tension and contraction can be generated by the controller and the display will indicate the progress achieved in the muscles being treated by the photo-thermal device 10. The degree of muscle tension and contraction indicated by the electrodes 63 will help establish if additional treatment time is necessary or if other treatment options may be necessary. The degree of muscle tension and contraction provides measurable information on the status of the treatment that can be used to supplement the subjective reactions of the user to the treatment received by the photo-thermal device 10. The electrodes 63 are disposed on the flexible circuit 37 in a manner that will provide effective information on the degree of tension and contraction in the muscles that are being treated by the photo-thermal device 10.

A cover or encapsulant 73 is positioned over the light transmitting diodes 43, resistors 53, thermistors 59 and electrodes 63 to separate these elements from the environment in which the photo-thermal device 10 is used. The cover is an infra-red transparent material which is flexible so that the cover can be displaced to accommodate the shape of the surface that is receiving the light and heat stimulation from the photo-thermal device 10. The cover 73 should be at least as flexible as the resilient material 27 and capable of being displaced in more than one direction to accommodate the displacement of the resilient material and electronic components that are positioned on the surface of the resilient material that extends from the opening 23. An example of an infra-red transparent material that can be satisfactorily used with the invention is D9930 doming material produced by Epic. The cover 73 engages the sidewall 17 of the outer housing 15 so that the components positioned in the cavity 19 are totally encapsulated by the cover. The cover 73 is also impervious to bacteria, viruses and debris provides a flexible barrier that separates the electrical components mounted on the flexible circuit 37 positioned in the resilient material 27 and protects the electronic components from environmental contaminants when the photo-thermal device 10 is utilized to provide light and heat stimulation. The cover 73 is formed of a material that can be cleaned with a 1% bleach solution and mild soap so that the surface of the device 10 that comes into contact with the user can be completely and adequately disinfected and protect the user from cross-contamination from other users or environmental contaminants. The cover 73 and the outer housing 15 can also be sterilized in cold sterilization processes if the photo-thermal device 10 is being used in a sterile environment.

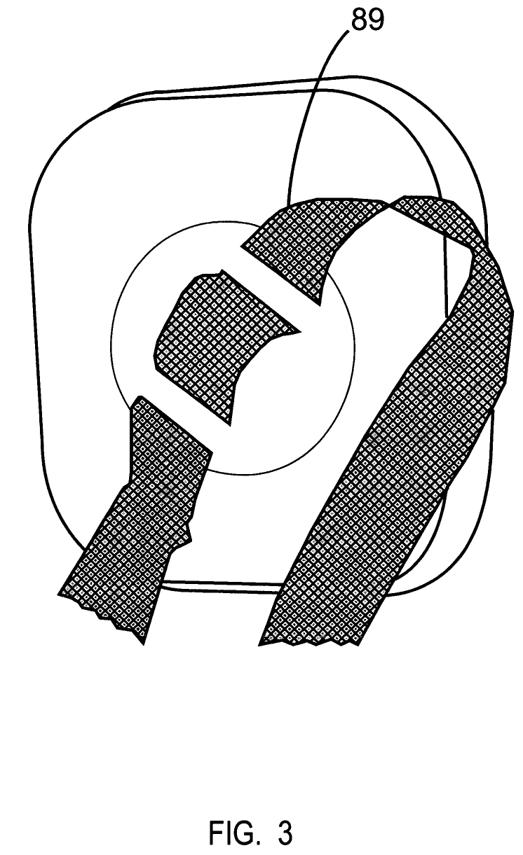

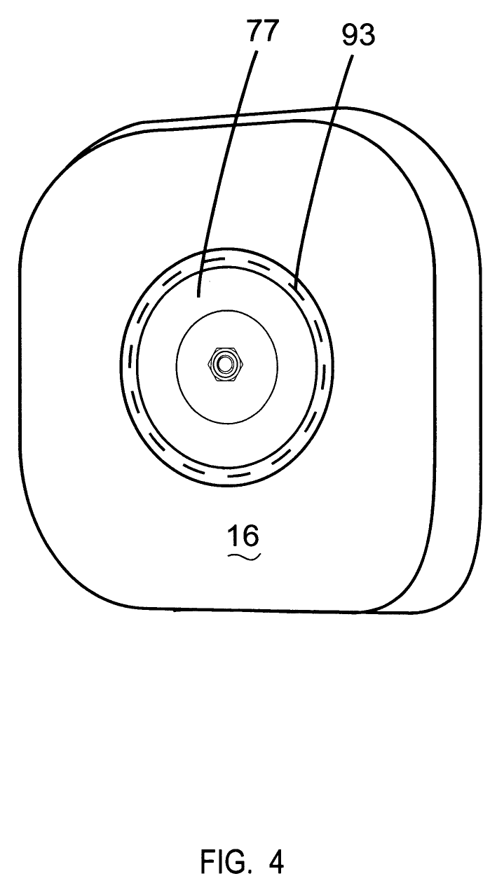

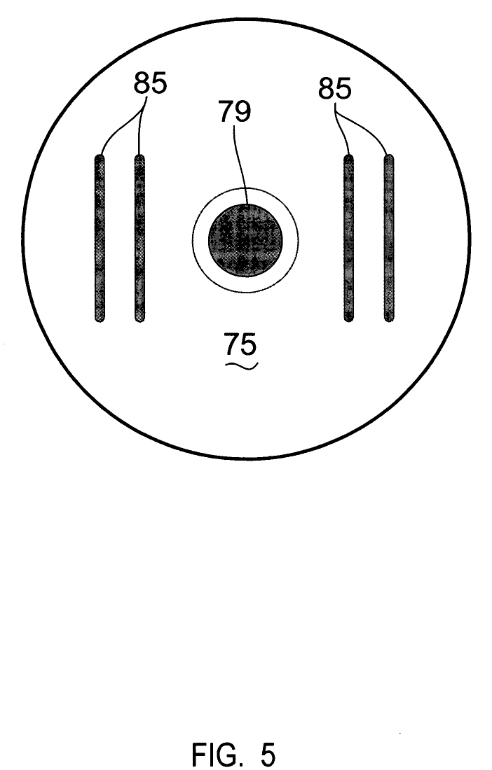

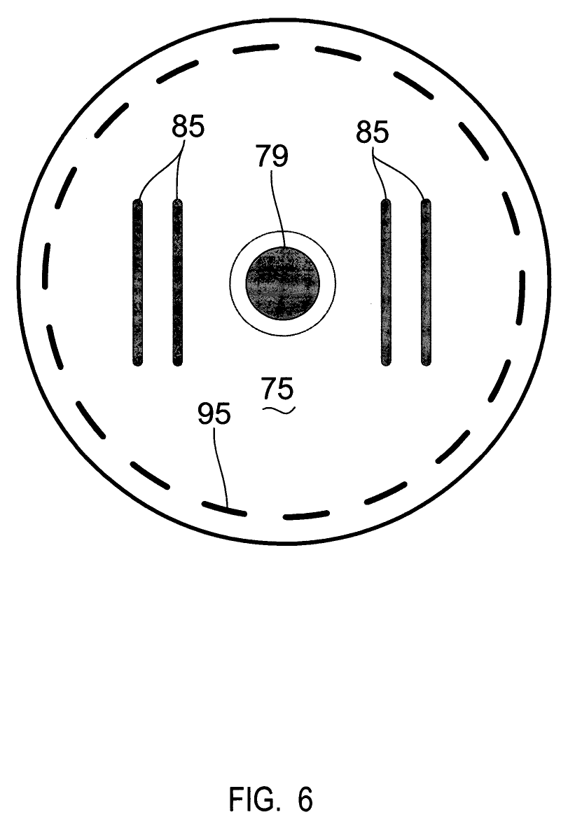

As shown in FIGS. 3-6 the back wall 16 of the device 10 can have a mechanism for securing the device to a patient during use. The securing mechanism is a generally circular disc 75 that is removably and rotatably positioned in a generally circular cavity 77 formed in the back wall 16 of the photo-thermal therapeutic device 10. The circular disc 75 has a hole 79 substantially in the center of the circular disc and the hole is disposed to receive bolt 81 that extends from the back wall 16. A nut 83 is positioned on the bolt 81 to secure the disc 75 to the photo-thermal therapeutic device 10. The circular disc 75 has a plurality of slots 85 located on either side of the hole 79. The slots 85 are disposed in substantially parallel relationship and the slots are designed to accept a strap 89 whereby the strap 89 can be positioned in the slots 85 in a well known manner to secure the strap to the circular disc 75. The strap 89 has a sufficient length and a suitable clasp or securing device associated with the strap wherein the strap can be utilized to maintain the photo-thermal therapeutic device in the desired location on a patient. A plurality of indentations 93 are positioned around the outer periphery of the cavity 77. The circular disc 75 has a plurality of projections 95 and the projections are disposed to be in alignment with the indentations 93 in the cavity 77. When the desired position for the circular disc and the strap 89 are determined, the nut 83 can be advanced on the bolt 81 to secure the circular disc 75 in the desired position with respect to the back wall 16 of the device 10. The projections 95 will also extend into the indentations 93 and assist in preventing rotation of the circular disc 75 with respect to the cavity 77 and back wall 16. As there are a plurality of indentations and projections it is possible to obtain the desired orientation for the circular disc 75 and still have engagement between the indentations 93 and the projections 95. If a different location for the circular disc 75 and the strap 89 is desired it is only necessary to release the pressure on the nut 83 to allow the circular disc 75 to be rotated to a new location. In the new location for the circular disc 75 the projections 95 will engage indentation 93 and assist in maintaining the circular disc 75 and strap 89 in the desired location when the nut 83 is tightened on the bolt 81. It is also possible to place just enough tension on the circular disc 75 with the nut 83 to allow the circular disc to be rotated without releasing or moving the nut. The interaction of the projections 95 and the indentations 93 will retain the circular disc in the desired new location with the retaining force of the nut 83.

In operation, the photo-thermal device 10 is positioned on the surface that is to receive light and heat stimulation. The cover 73, light emitting diodes 43, resistors 53, thermistors 59, electrodes 63 and resilient material 27 can deform to the convex and concave curvatures of the user of the photo-thermal device 10. The resilient nature of these components is sufficient to accommodate substantial and abrupt changes in contour to ensure maximum contact of the photo-thermal device 10 with the various areas of the user that are to receive light and heat stimulation. The resilient structure of the photo-thermal device enhances its ability to deliver photo and thermal energy more effectively to almost all surfaces.

As previously discussed, the photo-thermal device 10 has a controller 47 that can be operated in a manner to sequence the patterns in a rate of activation for the light emitting diodes 47 and resistors 53. Photo therapy when applied to the skin surface of the user produces a multiplicity of effects and nerve stimulation is one of these effects. The nerve fibers that carry sharp pain are identified as fibers. The A fibers are the largest, fastest conducting in fibers and are mylinated. Chronic pain is carried by the smaller, slower conducting and unmylinated C fibers. Both A and C fibers can accept rapid multiple stimulation up to a limit. When photo-thermal energy treatment is used to reduce pain the photo-thermal energy is directed at stimulating the A and C nerve fibers. When a nerve is subjected to a stimulus, the stimulus is carried along the course of the nerve. However, there is a limit to how much stimulation can be administered to a nerve before it fails to respond. Once the stimulation limit has been exceeded, the nerve must be allowed to recover before it can properly respond to additional stimulation. Studies have shown that a nerve can receive a stimulus and generate an effective response approximately 92 times a second. Ideally, to obtain the maximum desired clinical effect, a nerve should be stimulated rapidly enough to approach this limit but not rapidly enough to exceed this limit. Treatment with medical devices can frequently produce a condition known as adaptation where there is a progressive decrease in response to a treatment following a repetitive administration of such treatment. When adaptation occurs the treatment becomes less effective to ineffective. The photo-thermal device 10 of the present invention offers extensive flexibility in adjusting the treatment parameters to avoid the creation of an adaptation state where the user does not respond in the desired manner to the treatment. The photo-thermal device 10 has the ability to provide for adjustment of several parameters such as the duty cycle for the light emitting diodes, the frequencies for the light emitting diodes, the temperature of the surface that is being treated, the treatment time, sequencing patterns for the light emitting diodes and the rate at which each light emitting diode or clusters of light emitting diodes are electrically energized. All of the above parameters can be controlled to provide the most effective light and heat stimulation to treat the user of the photo-thermal device 10. The parameters of the photo-thermal device 10 can be preset to treat specific conditions in specific locations or the parameters can be individually controlled to create a custom treatment modality. The temperature control provided by the thermistors 59 substantially reduces the risk of thermal injury while delivering thermal energy to the area to be treated. Controlling the temperature of the surface that is to be treated is particularly important in dealing with patients that have some form of neuropathy that may limit the patient's ability to adequately sense the temperature of the surface where the treatment is being applied. The variable refresh rate provided by the controller 47 further allows the photo-thermal device 10 to be applied to a user in patterns of use that reduces adaptation by the user. If adaptation occurs this can reduce the effectiveness of the treatment.

The electrodes 63 also provide an electromyographic evaluation of the degree of muscle tension and contraction in the area that is being treated. The electromyographic evaluation provides useful information on the progress and effectiveness of the treatment to the selected area. With this information the user and any associated health care professional can evaluate, in an empirical manner, the effectiveness of the treatment.

The above description of the invention is given for the sake of explanation. Various substitutions and modifications, other than those cited, can be made without departing from the scope of the following claims.

We claim:

1. A photo-thermal device for applying light and heat stimulation to a surface comprising:
an outer housing, said outer housing defining a cavity having an opening;
a resilient material positioned in said cavity of said outer housing;
a plurality of light emitting diodes positioned in said resilient material; said light emitting diodes positioned in said resilient material adjacent said opening in said outer housing, whereby said resilient material allows said light emitting diodes to be displaced to accommodate the shape of the surface that is receiving light and heat stimulation;

2. The device of claim 1 wherein said resilient material is a foam material.

3. The device of claim 1.5 wherein the foam material is rubber.

4. The device of claim 1 wherein a circuit in said outer housing operative to energize said light emitting diodes.

5. The device of claim 2 wherein said circuit is flexible and moves with said light emitting diodes are displaced.

6. The device of claim 3 wherein said light emitting diodes and said circuit can be displaced in at least a vertical and a horizontal direction to accommodate the shape of the surface that is receiving light and heat stimulation.

7. The device of claim 1 wherein said plurality of light emitting diodes are encapsulated in a flexible and infra-red transparent material that can be cleaned using cold sterilization methods.

8. The device of claim 5 wherein the flexible and infra-red transparent encapsulation material is a fluid impervious polymer.

9. The device of claim 1 wherein the light emitting diodes emit light in the infra-red spectrum.

10. The device of claim 1 wherein said plurality of light emitting diodes are selectively energized, said selectively energizing of said light emitting diodes acting to inhibit adaptation to said stimulation and thereby enhance the effectiveness of said stimulation.

11. The device of claim 1 wherein at least one heating element is positioned in said resilient material adjacent said opening, said at least one heating element being selectively operable to augment the thermal treatment provided by the plurality of light emitting diodes.

12. The device of claim 9 wherein a flexible circuit is positioned in said outer housing to operatively energize said at least one heating element, said at least one heating element and said flexible circuit being capable of being displaced to accommodate the shape of the surface that is receiving light and heat stimulation.

13. The device of claim 10 wherein said at least one heating element is encapsulated in a flexible and infra-red transparent material that can be cleaned using cold sterilization methods.

14. The device of claim 10 wherein a plurality of heating elements are positioned in said resilient material, said heating elements being positioned adjacent said light emitting diodes.

15. The device of claim 11 wherein said heating elements can be displaced in a vertical and horizontal direction to accommodate the shape of the surface.

16. The device of claim 11 wherein said heating elements are resistive heating elements.

17. The device of claim 1 wherein at least one thermistor is positioned in said resilient material adjacent said opening in said outer housing, said at least one thermistor being disposed to monitor the temperature of the surface that is receiving light and heat stimulation, whereby said resilient material allows said thermistor to be displaced to accommodate the shape of the surface that is receiving light and heat stimulation.

18. The device of claim 13 wherein a flexible circuit is positioned in said outer housing to operatively control said at least one thermistor. The device of claim 14 wherein a plurality of thermistors are positioned in the resilient material.

19. The device of claim 14 wherein a plurality of thermistors are positioned in the resilient material.

20. The device of claim 14 wherein the flexible circuit provides thermal feedback loop that measure the temperature on the surface and to compare this temperature with a preselected desired temperature range and controlling the energy supplied to the at least one light emitting diode to maintain the temperature on said surface in said preselected desired temperature range.

21. The device of claim 16 wherein at least one heating element is positioned in said resilient material and said at least one heating element is operatively connected to said thermal feedback loop whereby the energy supplied to said at least one heating element can be controlled to maintain temperature on said surface, as measured by said at least one thermistor, in said preselected desired range.

22. The device of claim 17 wherein a plurality of heating elements are positioned in the resilient material and operatively connected to said thermal feedback loop.

23. The device of claim 18 wherein said resilient material allows said plurality of heating elements to be displaced to accommodate the shape of the surface that is receiving light and heat stimulation.

24. The device of claim 1 wherein at least one EMG electrode is positioned in said resilient material adjacent said opening in said outer housing, said at least one EMG electrode being disposed to monitor the muscle tension and contraction of the surface that is receiving light and heat stimulation, whereby said resilient material allows said EMG electrode to be displaced to accommodate the shape of the surface that is receiving light and heat stimulation.

25. The device of claim 20 wherein a flexible circuit is positioned in said outer housing to operatively control said at least one EMG electrode.

26. The device of claim 20 wherein a plurality of EMG electrodes, are positioned in the resilient material.
